(19) United States
(12) Patent Application Publication
(10) Pub. No.: US 2014/0303663 A1
(43) Pub. Date: Oct. 9, 2014

(54) APPARATUS AND METHOD FOR VASCULAR AND NERVE SEPARATION AND BRIDGING

(71) Applicant: Vanderbilt University, Nashville, TN (US)

(72) Inventors: Karen Joos, Nashville, TN (US); Jin Shen, Nashville, TN (US); Nabil Simaan, Nashville, TN (US)

(21) Appl. No.: 14/353,444

(22) PCT Filed: Oct. 25, 2012
(86) PCT No.: PCT/US12/61849

§ 371 Date: Apr. 22, 2014

Related U.S. Application Data
(60) Provisional application No. 61/551,102, filed on Oct. 25, 2011.

(51) Int. Cl. A61F 9/007; A61M 29/00; A61B 17/02

(52) U.S. Cl. CPC A61F 9/007; A61B 17/0231; A61M 29/00
USPC 606/191

(57) ABSTRACT

Devices, methods, tools, and kits for surgically separating two pressure-sensitive vessels (e.g., arteriole, vein, and/or nerve) at a point of contact or within about 1 mm of the contact. The device includes a biocompatible sheet of material, such as a bridge or separator or external stent. The device is positioned between one or more pressure-sensitive vessels or nerves to alleviate compression with a the tool includes a deployment mechanism and a user interface (e.g., a controller or robot) for inserting the device between the two pressure-sensitive vessels or nerves.

### RELATED APPLICATIONS

This application is a non-provisional application of and claims priority to U.S. Provisional Patent Application No. 61/551,102, filed on Oct. 25, 2011, the entire contents of which are incorporated herein by reference.

### BACKGROUND OF THE INVENTION

Hemorrhaging and vision loss can occur from a branch retinal vein occlusion (BRVO) where an arteriole passes over a vein to restrict the passage of blood flow. Retinal vascular disease is a leading cause of blindness. BRVO is the second most common retinal vascular disorder following diabetic retinopathy. Population-based studies reflect an overall adult prevalence of 4.42 per 1000 people or 13.9 million people worldwide with BRVO (R. L. McIntosh et al., “Interventions for branch retinal vein occlusion: an evidence-based systematic review.,” Ophthalmology, vol. 114, pp. 835-854, 2007) and occurrence increases with age. BRVO can cause a decrease in vision due to ischemia or edema of the macula, and/or vitreous hemorrhage. More than half of patients with BRVO develop visual acuity worse than 20/40. BRVO typically occurs at arteriovenous crossing sites with the artery positioned anterior to the vein producing compression (G. T. Frangieh et al., “Histopathologic study of nine branch retinal vein occlusions.,” Arch Ophthalmol., vol. 100, pp. 1132-1140, 1982). The Branch Vein Occlusion Study (Anonymous, “Argon laser photocoagulation for macular edema in branch vein occlusion. The Branch Vein Occlusion Study Group,” Am J Ophthalmol, vol. 98, pp. 271-82, 1984) and the Standard Care versus Corticosteroid for Retinal Vein Occlusion Study (I. M. Scott IU et al.; SCORE Study Research Group., “A randomized trial comparing the efficacy and safety of intravitreal triamcinolone with standard care to treat vision loss associated with macular Edema secondary to branch retinal vein occlusion: the Standard Care vs Corticosteroid for Retinal Vein Occlusion (SCORE) study report 6.,” Arch Ophthalmol, vol. 127, pp. 1115-28, 2009) demonstrated that grid laser is helpful for resolving macular edema. Alternatives have been sought because retinal hemorrhages interfere with laser treatment and laser scars can decrease vision. Medical therapies include intravitreal injection of corticosteroids or VEGF inhibitors to treat the retinal edema rather than the underlying blood flow obstruction. However, a significant number of patients are unresponsive to medical therapy and retain macular edema and poor vision.

The first report of surgical decompression as a successful potential treatment for BRVO with a vitrectomy and technically challenging separation of the common adventitial sheath of the crossing artery and vein (sheathotomy) was published in 1988 by Osterloh and Charles. C. S. Osterloh M D, “Surgical decompression of branch retinal vein occlusions,” Arch Ophthalmol., vol. 106, pp. 1469-71, 1988. Multiple reports have suggested that vitrectomy with sheathotomy may improve vision in patients with recalcitrant macular edema unresponsive to laser therapy and/or medical therapy. J. Mason et al., “Sheathotomy to decompress branch retinal vein occlusion: a matched control study,” Ophthalmology, vol. 111, pp. 540-545, 2004; U. Mester and P. Dillinger, “Vitrectomy with arteriovenous decompression and internal limiting membrane dissection in branch retinal vein occlusion,” Retina, vol. 22, pp. 740-746, 2002; I. K. Oh et al., “ Long-term visual outcome of arteriovenous adventitial sheathotomy on branch retinal vein occlusion induced macular edema,” Korean J Ophthalmol, vol. 22, pp. 1-5, 2008; B. R. Opremcak E M, “Surgical decompression of branch retinal vein occlusion via arteriovenous crossing sheathotomy: a prospective review of 15 cases,” Retina, vol. 19, pp. 1-5, 1999; N. Rodanant and S. Tjoongsuwan, “Sheathotomy without separation of venule overlying arteriole at occlusion site in uncommon branch retinal vein occlusion,” J Med Assoc Thai., vol. 88, pp. 143-150, 2005; S. Yamamoto et al., “Vitrectomy with or without arteriovenous adventitial sheathotomy for macular edema associated with branch retinal vein occlusion,” Am J Ophthalmol, vol. 138, pp. 907-914, 2004; J. C. Hwang et al., “ Combined arteriovenous sheathotomy and intraoperative intravitreal triamcinolone acetonide for branch retinal vein occlusion,” Br J Ophthalmol, vol. 94, pp. 1483-1489, 2010; G. Shah, “Adventitial sheathotomy for treatment of macular edema associated with branch retinal vein occlusion.,” Curr Opin Ophthalmol, vol. 11, pp. 171-4, 2000.

One study reported no difference between sheathotomy versus intravitreal triamcinolone acetonide injection, but they did not limit their subjects to medically recalcitrant edema (E. J. Chung et al., “ Arteriovenous crossing sheathotomy versus intravitreal triamcinolone acetonide injection for treatment of macular edema associated with branch retinal vein occlusion,38 Graefes Arch Clin Exp Ophthalmol, vol. 246, pp. 967-974, 2008). Because a one-year course of anti-VEGF ranibizumab may exceed $23,000, (W. E. Smiddy, “ Economic Considerations of Macular Edema Therapies,” Ophthalmology, 2011) the cost of a highly successful surgical intervention could be cost-effective in BRVO treatment. Precise robotic control would likely reduce iatrogenic surgical complications of vitreous hemorrhage (B. R. Opremcak E M, “Surgical decompression of branch retinal vein occlusion via arteriovenous crossing sheathotomy: a prospective review of 15 cases,” Retina, vol. 19, pp. 1-5, 1999) or localized retinal detachment (M. T. Cahil et al., “The effect of arteriovenous sheathotomy on cystoid macular oedema secondary to branch retinal vein occlusion,” Br J Ophthalmol, vol. 87, pp. 1329-1332, 2003) at the arteriovenous sheathotomy site.

### SUMMARY OF THE INVENTION

The invention relates to devices, methods, tools, and kits for surgically separating two pressure-sensitive vessels (e.g., an arteriole and a vein) or two nerves or a pressure-sensitive vessel and a nerve at a point of contact or within about 1 mm of the contact. In particular, the invention is directed to minimally invasive micro-surgery of the eye targeting micro blood vessels with characteristic dimensions ranging from about 10-400 μm in diameter.

The invention also relates to a device comprising a biocompatible sheet of material, such as a bridge or separator or external stent, that is configured for placement between the pressure-sensitive vessels or nerves to permit adequate flow through the vessels and to alleviate any compression. The device is precisely positioned between the pressure-sensitive vessels or nerves with the use of an instrument or tool.

The tool includes a user interface end (e.g., proximal end) and a working end (e.g., distal end). The device is releasably coupled to the working end, which is inserted into an anatomical target (e.g., eye) to position the device. The user interface can include a microsurgical robotic system that is manipulated by the user for positioning the working end and the device.

A kit can include one or more instruments and one or more devices (e.g., biocompatible separators or bridges or external stents) and/or a bridge inserter to place between the vessels or nerves, to surgically separate the vessels or nerves. The placement procedure can be enhanced with optical coherence tomography (OCT) visualization and robotic micromanipulation to place the device.

In one embodiment, the present invention provides a medical device comprising a biocompatible sheet of material configured for insertion between a first pressure sensitive vessel and a second pressure sensitive vessel.

In another embodiment, the present invention provides a medical device comprising a biocompatible sheet of material configured for insertion between a pressure sensitive vessel and a nerve.

In yet another embodiment, the present invention provides a medical device comprising a biocompatible sheet of material configured for insertion between a first nerve and a second nerve.

The invention also provides a method for separating two components in a patient. The method comprises inserting a device through a lumen in the patient, separating a first pressure sensitive vessel from a second pressure sensitive vessel with the device to create an opening, and inserting a biocompatible sheet of material into the opening to maintain separation of at least a portion of the first pressure sensitive vessel and the second pressure sensitive vessel.

The invention also provides a method for separating two components in a patient. The method comprises inserting a device through a lumen in the patient; separating a first pressure sensitive vessel from a nerve with the device to create an opening, and inserting a biocompatible sheet of material into the opening.

The invention also provides a method for separating two components in a patient. The method comprises inserting a device through a lumen in the patient, separating a first nerve from a second nerve with the device to create an opening, and inserting a biocompatible sheet of material into the opening.

The invention also provides a tool for positioning a device to separate two components. The tool comprises a first tube in communication with a user interface, a second flexible tube coupled to and in a telescoping relationship with the first tube, and a third tube coupled to and in a telescoping relationship with the second flexible tube, the device coupled to an outer surface of the third tube, and wherein the device is positioned at least partially between the two components when the third tube is retracted into the second flexible tube.

The invention also provides a tool for positioning a device to separate two components according to another embodiment. The tool comprises a first tube in communication with a user interface, a second flexible tube coupled to and in a telescoping relationship with the first tube, and a wire coupled to and in telescoping relationship with the second flexible tube, the wire including a deployment section at a distal end thereof, the device coupled to an outer surface of the wire, and wherein the device is configured to slide over the deployment section and onto at least one of the components when the second flexible tube pushes the device over the expansion segment.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein are for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings.

Although directional references, such as upper, lower, downward, upward, rearward, bottom, front, rear, etc., may be made herein in describing the drawings, these references are made relative to the drawings (as normally viewed) for convenience. These directions are not intended to be taken literally or limit the present invention in any form. In addition, terms such as “first,” “second,” and “third” are used herein for purposes of description and are not intended to indicate or imply relative importance or significance.

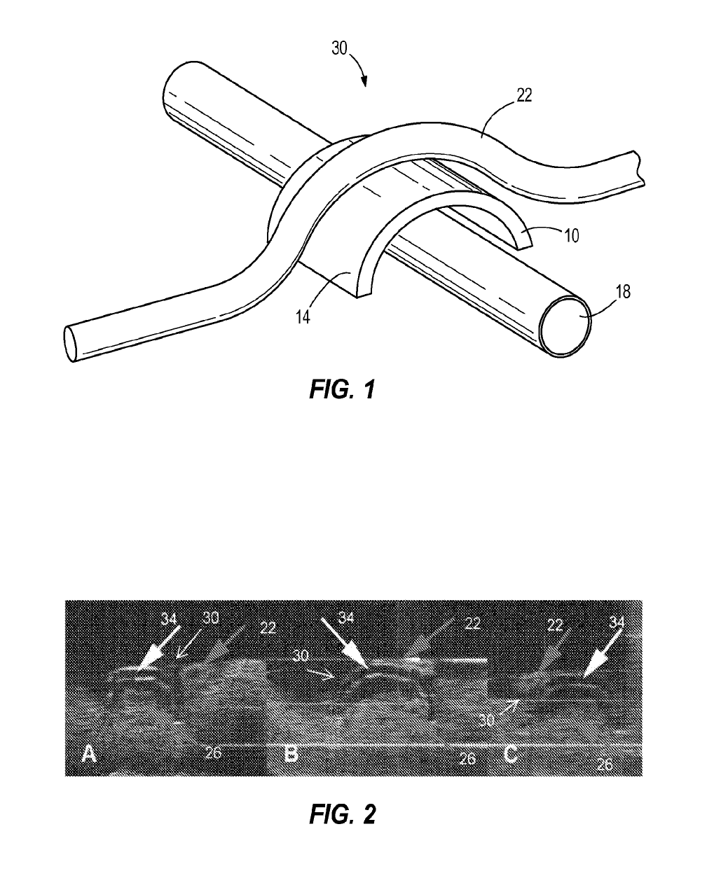

FIG. 1 schematically illustrates a device 10 for separating two pressure sensitive vessels or two nerves or a pressure-sensitive vessel and a nerve according to one embodiment of the present invention. The device 10 includes a sheet of material 14 configured to be positioned between a first pressure sensitive vessel, such as a vein or arteriole, and a second pressure sensitive vessel, such as a vein or arteriole or between two nerves or between a pressure-sensitive vessel and a nerve. As illustrated in FIG. 1, the device 10 is positioned between a vein 18 and an arteriole 22 at a crossing 30 (e.g., a location where the vein and arteriole overlap or touch one another). The sheet of material 14 can be flexible or pre-formed. The sheet of material 14 can include a plurality of individual segments that are fused or coupled together that allow the sheet to be flexible and thus able to be manipulated into different configurations depending on the location of use. In some alternative embodiments, the sheet of material 14 is in the form of a half-cylinder. The sheet of material 14 can comprise any suitable material or combinations of materials that are biocompatible with human tissue, such as the retina, including but not limited to one of a super-elastic alloy, a shape memory alloy, and a smart material. In some alternative embodiments, the sheet of material 14 can include nickel titanium (NiTi), ionic polymer metal composite (IPMC) or poly(methyl methacrylate) (PMMA). Additionally, the sheet of material 14 can include drugs or medications embedded therein that elute from the material(s) over time.

The devices, tools, methods and kits of this invention can be used for similar procedures in other systems in the body for the separation of vessels and/or nerves causing compression or compromised flow. For example, this could include vascular decompression of the trigeminal nerve in trigeminal neuralgia.

In one example, a retinal venule 26 and arterial 22 crossing 30 was separated in a cadaver pig eye (see FIG. 2). A prototype half-cylinder sheet of material 34 was inserted under the arteriole 38 and over the retinal venule 26. The position of the half-cylinder sheet of material 34 was imaged by OCT (see FIG. 2). FIG. 2 shows the half-cylinder sheet of material 34 which is positioned over a cross-section profile of a retinal venule 26 and under a retinal artery 22.

The device 10 can be in the form of external stents or bridges and they can be created using the following technologies:

Super-elastic nickel titanium (NiTi) stents are pre-shaped to wrap over the blood vessel. These stents use a deployment mechanism that supports them in an expanded configuration and provides gradual release around the blood vessel. These stents can also be pre-shaped to a specific diameter based on imaging of the patient's eye and segmentation of these images to determine the blood vessel size. The pre-shaping process involves wrapping a sheet of NiTi around a wire with a diameter matching the blood vessel then heating the device to about 400-500 degrees Celsius for an hour and then cooling down the device to set its shape.

Electro-active polymer composites (e.g., ionic polymer metal composite; IPMC) stents can be used in the form of a Nafion strip that curls into shape upon activation. These stents are made of a layer of Nafion between a cathode and an anode layer, upon activation of about 1-2 Volts differential in an aqueous environment the ions are transported to one side of the polymer and cause it to swell and bend. Upon release of voltage these polymers retain their shape.

A biocompatible polymer such as PMMA pre-formed in a sloping bridge configuration to maintain separation.

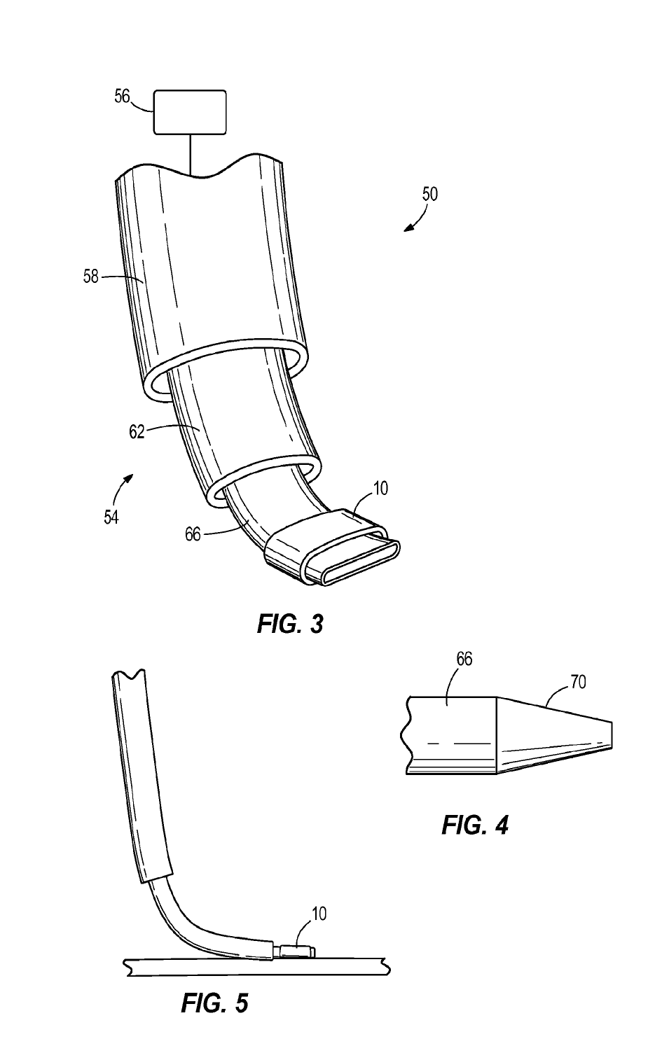

The device 10 is inserted through a lumen in a patient with an apparatus 50, which includes a deployment mechanism 54 and a user interface such as an external controller 56 (e.g., robot). FIGS. 3-5 illustrate one embodiment of a deployment mechanism 54 for external devices 10. The deployment mechanism 54 includes a support tube 58 that is coupled to the controller 56, a second tube 62, and a deployment tab 66. The support tube 58 comprises stainless steel and/or a polymer and is substantially more rigid than the part it holds. The second tube 62 is configured to be received within the support tube 58 and is steerable and bendable. The second tube 62 is independently controlled by the controller 56 and can telescope with respect to the support tube 58 thereby changing length and deployment angle. This second tube 62 can comprise a super elastic NiTi material that is pre-shaped to bend its tip in a circular arc with a predetermined radius. By extending the second tube 62 out of the support tube 58, the approach angle for deployment of the device 10 is controlled. The deployment tab 66 is configured to be received within the second tube 62 and holds the device 10 in an open and/or extended position. The deployment tab 66 is also independently controlled by the controller 56 and can telescope with respect to the support tube 58 and the second tube 62. The deployment tab 66 includes a distal end having a tapered tip 70 such that when the deployment tab 66 is retracted into the second tube 62, the device 10 slides off and the distal end of the device 10 starts curling around to at least partially surround or conform to the outer diameter of the blood vessel to thereby separate the vessel from another vessel or nerve 18, 22.

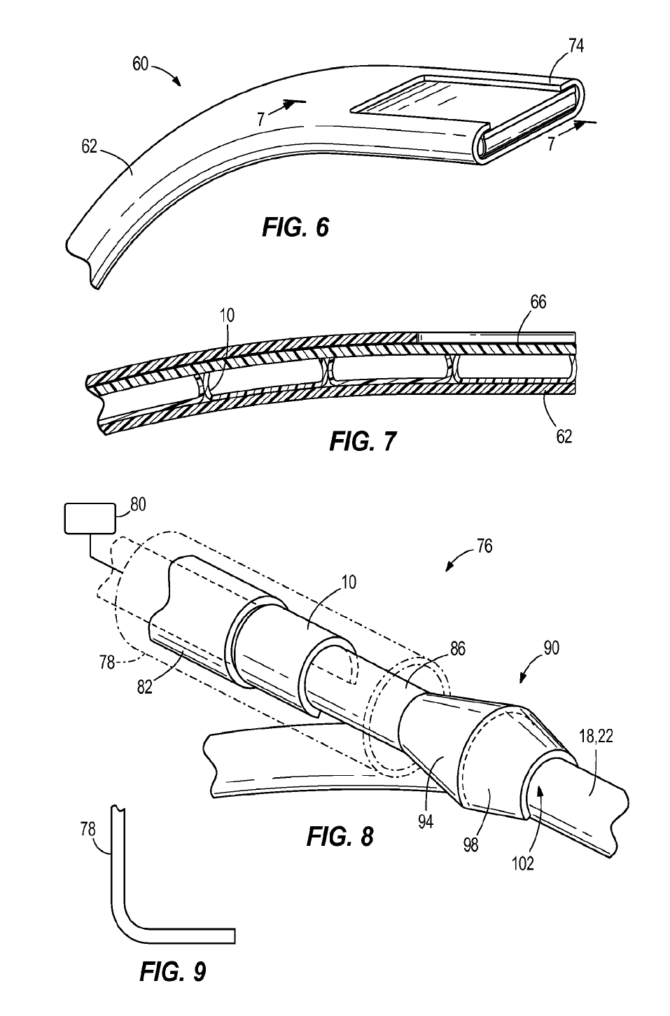

FIGS. 6-7 illustrate a second embodiment of a deployment mechanism 60. This second embodiment utilizes a similar arrangement with the deployment tab 66, second tube 62, support tube 58, and controller 56 as illustrated in FIGS. 3-5 of the first embodiment of the deployment mechanism 54. The deployment mechanism 60 includes deployment tab 66 configured to support a plurality of devices 10. The plurality of devices 10 are arranged on the deployment tab 66 with their longitudinal axis perpendicular to the backbone of the second tube 62. The deployment tab 66 in this second embodiment includes a release slot 74 in the second tube 62. The devices 10 are arranged serially in an extended configuration as shown in FIG. 7. The deployment tab 66 holds the devices 10 in an extended (open) configuration. When the deployment tab 66 is retracted, the distal end of each of the devices 10 curls gradually around the blood vessel thereby separating the vessel from another vessel or nerve 18, 22.

One difference between the second embodiment of the deployment mechanism 60 and the first embodiment of the deployment mechanism 54 is that the second embodiment allows for approaching the target blood vessel where the plane containing the second tube 62 is generally perpendicular to the target blood vessel. In the first embodiment of the deployment mechanism, this plane contains the blood vessel. The second embodiment of the deployment mechanism also allows for continuous release of multiple devices 10 while the first embodiment allows for deployment of a pre-loaded device 10 (e.g., a single use tip).

FIGS. 8-9 illustrate a third embodiment of a deployment mechanism 76 coupled to a controller 80 (e.g., robot). The deployment mechanism 76 in this embodiment includes a pre-bent support tube 78 that allows for adjustment of its distal tip location inside the eye or other target area. The deployment mechanism 76 also includes a second tube 82 and a deployment tab 86. The second tube 82 is configured to be received within the support tube 78 and is steerable and bendable. The second tube 82 is independently controlled by the controller 80 and can telescope with respect to the support tube 78 thereby changing length and deployment angle.

The deployment tab 86 is generally configured as a conduit or wire with a deployment section 90 at its distal end. The deployment tab 86 is configured to be at least partially received within the second tube 82. The deployment tab 86 is independently controlled by the controller 80 and can telescope with respect to the support tube 78 and the second tube 82. The deployment section 90 includes a first segment 94 (e.g., expansion segment) where a width (or diameter) of the first segment gradually increases from a proximal end to a mid-section and a second segment 98 (e.g., gradual release segment) where a width (or diameter) of the second segment gradually decreases from the mid-section to a distal end. The deployment section 90 serves a dual-purpose of expanding the device 10 and gradually releasing it to surround or conform to the blood vessel. The release of the device 10 is controlled by a gradual pushing of the second tube 82 in order to gradually advance the device 10 along the axis of the deployment tab 86. The deployment section 90 includes a recessed area 102 configured to receive a blood vessel such that the axis of the deployment tab 86 is substantially coaxial with the axis of at least a portion of the blood vessel. As the device 10 is advanced along the deployment tab 86 and the deployment section 90, the device 10 gradually expands as it traverses along the first segment 94 of the deployment section 90. As the device 10 continues along the deployment section 90, the device 10 is positioned on the blood vessel (or nerve) as it gradually slides off of the second segment 98 of the deployment section 90. The device 10 then gradually contours or at least partially surrounds an external surface of the target blood vessel thereby separating the vessel from another vessel or nerve 18, 22. The second tube 82 can include longitudinal slots that allow a distal end to elastically expand as it pushes the device 10 over or along the deployment section 90.

Various features of the invention are set forth in the following claims.

What is claimed is:

1. A medical device comprising:
a biocompatible sheet of material configured for insertion between a first pressure sensitive vessel and a second pressure sensitive vessel.

2. The medical device of claim 1, wherein the sheet of material is pre-formed.

3. The medical device of claim 1, wherein the sheet of material is actively conformed to one of the first pressure sensitive vessel and the second pressure sensitive vessel during placement.

4. The medical device of claim 1, wherein the biocompatible sheet of material comprises one of a super-elastic alloy, a shape memory alloy, a smart material and a biocompatible polymer.

5. The medical device of claim 1, wherein the biocompatible sheet of material comprises one of NiTi, IPMC and PMMA.

6. The medical device of claim 1, wherein the biocompatible sheet of material is shaped in the form of a half-cylinder.

7. The medical device of claim 1, wherein the biocompatible sheet of material is shaped in the form of a cylindrical section.

8. The medical device of claim 1, wherein the first pressure sensitive vessel is positioned over the second pressure sensitive vessel.

9. The medical device of claim 1, wherein the first pressure sensitive vessel is positioned on either side of the second pressure sensitive vessel.

10. A medical device comprising:
a biocompatible sheet of material configured for insertion between a pressure sensitive vessel and a nerve.

11. A medical device comprising:
a biocompatible sheet of material configured for insertion between a first nerve and a second nerve.

12. A method for separating two components in a patient, the method comprising:
inserting a device through a lumen in the patient;
separating a first pressure sensitive vessel from a second pressure sensitive vessel with the device to create an opening; and
inserting a biocompatible sheet of material into the opening to maintain separation of at least a portion of the first pressure sensitive vessel and the second pressure sensitive vessel.

13. A method for separating two components in a patient, the method comprising:
inserting a device through a lumen in the patient;
separating a first pressure sensitive vessel from a nerve with the device to create an opening; and
inserting a biocompatible sheet of material into the opening.

14. A method for separating two components in a patient, the method comprising:
inserting a device through a lumen in the patient;
separating a first nerve from a second nerve with the device to create an opening; and
inserting a biocompatible sheet of material into the opening.

15. A tool for positioning a device to separate two components, the tool comprising:
a first tube in communication with a user interface;
a second flexible tube coupled to and in a telescoping relationship with the first tube; and
a third tube coupled to and in a telescoping relationship with the second flexible tube,
the device coupled to an outer surface of the third tube, and
wherein the device is positioned at least partially between the two components when the third tube is retracted into the second flexible tube.

16. A tool for positioning a device to separate two components, the tool comprising:
a first tube in communication with a user interface;
a second flexible tube coupled to and in a telescoping relationship with the first tube; and
a wire coupled to and in telescoping relationship with the second flexible tube, the wire including a deployment section at a distal end thereof, the device coupled to an outer surface of the wire, and
wherein the device is configured to slide over the deployment section and onto at least one of the components when the second flexible tube pushes the device over the expansion segment.

17. The tool of claim 16 wherein the deployment section includes a first segment having a gradually increasing width from a proximal end to a distal end and a second segment having a gradually decreasing width from a proximal end to a distal.

18. The tool of claim 16 wherein the deployment section includes a recess configured to receive at least a portion of one of the components thereby aligning a longitudinal axis of the wire with a longitudinal axis of the component.

19. The tool of claim 16 wherein the device is configured to conform to an outer surface of one of the components as it is positioned thereon.
